(19) United States
(12) Patent Application Publication
(10) Pub. No.: US 2026/0266692 A1
(43) Pub. Date: Sep. 10, 2026

(54) METHOD FOR CONSTRUCTING A BEARING BENCH TEST SPECTRUM BASED ON A COMBINED ROTATIONAL SPEED-TORQUE COUNTING METHOD

(71) Applicant: CHANG'AN UNIVERSITY, Xi'an (CN)

(72) Inventors: Xuan ZHAO, Xi'an (CN); Xiaolei YUAN, Xi'an (CN); Shu WANG, Xi'an (CN); Yisong CHEN, Xi'an (CN); Xuyue CHEN, Xi'an (CN); Sheng ZHU, Xi'an (CN)

(21) Appl. No.: 19/661,780

(22) Filed: Apr. 29, 2026

Related U.S. Application Data
(63) Continuation of application No. PCT/CN2025/114118, filed on Aug. 12, 2025.

(30) Foreign Application Priority Data
Sep. 13, 2024 (CN) 202411284736.6

(51) Int. Cl. G01M 13/045

(52) U.S. Cl. CPC G01M 13/045

(57) ABSTRACT

A method for constructing a bearing bench test spectrum based on a combined rotational speed-torque counting method comprises: step 1: acquiring and preprocessing sample load spectrum road operation data; step 2: performing combined rotational speed-torque counting on preprocessed data; step 3: constructing a bearing damage model; step 4: performing extrapolation on preprocessed data; and step 5: compiling a multi-level program load spectrum.

### CROSS-REFERENCE TO RELATED APPLICATIONS

This application is a continuation of International Patent Application No. PCT/CN2025/114118, filed on Aug. 12, 2025, which claims the benefit of priority from Chinese Patent Application No. 202411284736.6, filed on Sep. 13, 2024. The content of the aforementioned application, including any intervening amendments made thereto, is incorporated herein by reference in its entirety.

### TECHNICAL FIELD

This application relates to reliability and durability testing for automotive bearing components, and more particularly to a method for constructing a bearing bench test spectrum based on a combined rotational speed-torque counting method.

### BACKGROUND

In automotive applications, approximately over 90% of fractures can be attributed to fatigue failure of components. Fatigue failure is a common and highly hazardous failure mode for automotive parts. Fatigue damage predominantly occurs in components subjected to alternating loads, such as frame parts, leaf springs, coil springs, shafts, and rod-type components. Consequently, the importance of conducting reliability and durability analysis on the fatigue life of vehicle components during the forward-development research of vehicles cannot be underestimated.

In the process of vehicle reliability and durability analysis, the load spectrum serves as the top-level input for the entire forward durability development workflow. If the load input results deviate statistically from actual user loads, it can significantly impact subsequent design and analysis, leading to over-design or under-assessment issues. Therefore, load spectrum analysis is a crucial aspect of vehicle durability development. Conventional methods for analyzing reliability and durability require obtaining the service life from the full-cycle load spectra. However, the amount of data collected from test road load spectra cannot cover the entire load history across the entire lifecycle. Therefore, it is necessary to use the test load samples to infer the extreme loads and their frequencies that may occur within the full lifecycle.

As a key method for obtaining load distribution, counting methods are particularly important. For non-rotating components or shaft-type parts where torque fluctuations are independent of their rotational motion, the rainflow counting method can be applied. For parts like bearings whose rotational speed influences the fluctuation frequency, methods such as the rotating rainflow counting method or the rotating torque histogram counting method are required.

However, the aforementioned two methods cannot adequately reflect the joint distribution characteristics of rotational speed and torque, and fail to intuitively and efficiently demonstrate the impact of rotational speed and torque on bearing fatigue life. Therefore, this application emphasizes the accuracy in jointly considering rotational speed and torque when selecting load counting methods. Consequently, the combined rotational speed-torque counting method is adopted for bearing load counting, and load extrapolation is performed using Gaussian kernel function combined with the K strengthening coefficient extrapolation. This approach establishes a test spectrum construction method that provides significant guidance for analyzing bearing reliability and durability.

### SUMMARY

The objective of this application is to provide a method for constructing a bearing bench test spectrum based on a combined rotational speed-torque counting method. This application employs a frequency extrapolation method utilizing Gaussian kernel functions combined with K strengthening coefficient to enhance the authenticity of the bearing load spectrum data after extrapolation. Furthermore, since traditional bearing force analysis lacks sufficient consideration of factors such as bearing radial deformation, this application employs software simulation followed by piecewise interpolation to calculate bearing forces, thereby improving the accuracy of force analysis results. Additionally, considering that the current bearing test spectrum construction lacks the analysis of the impact of rotational speed on the constructed results, and rotational speed directly determines the values on fatigue life curves, this application proposes a load spectrum construction method based on combined rotational speed-torque counting method according to the correlation between rotational speed and bearing fatigue life. This method improves the accuracy of fatigue life analysis while intuitively reflecting the characteristics of load spectrum analysis by constructing multi-level spectrum images of the combined rotational speed-torque distribution.

Technical solutions of the present disclosure are described as follows.

This application provides a method for constructing a bearing bench test spectrum based on a combined rotational speed-torque counting method, comprising:
step 1: acquiring sample load spectrum road operation data and performing preprocessing, and extracting preprocessed time-domain data;
step 2: performing combined rotational speed-torque counting on the preprocessed time-domain data: recording loading frequencies under each rotational speed-torque interval through interval counting;
step 3: constructing a bearing damage model: conducting bearing force analysis, calculating an equivalent dynamic load based on analysis results, and combining a correction coefficient a_{ISO} considering rotational speed parameters to obtain a bearing life calculation formula with bearing torque and bearing rotational speed as independent variables, and combining joint counting results to establish the bearing damage model;
step 4: performing extrapolation on preprocessed data: based on results of the bearing damage model and extrapolated bearing target damage, obtaining a K strengthening coefficient, deriving a probability density function via kernel density estimation, and extrapolating counting results obtained in step 2 by Gaussian kernel function with the K strengthening coefficient to obtain an extrapolated frequency matrix for torque and rotational speed; and
step 5: compiling a multi-level program load spectrum: dividing each of the torque and rotational speed into eight levels according to specific ratios; applying a Palmgren-Miner rule in conjunction with a material S-N curve to the extrapolated rotational speed-torque frequency matrix from step 4, converting, by an upward equivalence method, frequencies within a 128×128 level interval to frequencies on a 8×8 level interval based on an equivalent damage conversion principle to obtain a 8×8 level bearing bench test spectrum for torque and rotational speed.

In some embodiments, the preprocessing in step 1 comprises outlier removal, threshold exceeding processing, and data resampling;
the outlier removal comprises: performing Fourier transform on original data to obtain an original frequency spectrum, filtering through Butterworth filter to screen torque data under each amplitude based on a frequency size, following classifying into a primary wave, a secondary wave, and a tertiary wave; and removing tertiary waves with amplitude data segments less than ±1 Nm to preliminarily filter out invalid waves;
the threshold exceeding processing is to fill missing data using piecewise interpolation after performing the outlier removal; and
the data resampling comprises: resampling by interpolation at a specified frequency over time, extracting non-repeating time points, and interpolating at a fixed, uniform sampling frequency to obtain the preprocessed time-domain data.

In some embodiments, during the combined rotational speed-torque counting in step 2, the preprocessed time-domain data is first divided into 128×128 level small intervals according to a target rotational speed-torque interval for counting, a rotational speed n(t) within a i-th rotational speed-torque small interval is integrated over time t, time for each interval is Δₜ_{ᵢ}, and the number of torque segments within each small interval is m, an integration result is recorded as a frequency Nᵢ for this interval, and frequencies for all intervals are stored as a matrix:

$${N}_{i} = {\sum }_{j = 1}^{m} {\int }_{{Δ}_{{t}_{i}}} n ( t ) d t .$$

In some embodiments, in the process of constructing the bearing damage model in step 3, a bearing damage D is calculated by a formula:

$$D = \frac{N}{{L}_{n m}} ;$$

wherein N represents a loading frequency at each level, and Lₙₘ represents a corresponding bearing life at each level, which is known.

In some embodiments, the loading frequencies corresponding to each rotational speed-torque level before extrapolation are obtained from step 2;
a bearing life before extrapolation:

$${L}_{n m} = {a}_{1} * {{a}_{ISO} ( \frac{{c}_{r}}{P} )}^{m} ;$$

wherein Cᵣ is a basic dynamic load rating; m is a bearing life exponent, for a ball bearing, m=3, and for a roller bearing,

$$m = \frac{10}{3} ;$$

a¹ is a reliability correction coefficient; P is an equivalent dynamic load; the correction coefficient a_{ISO} is a function of rotational speed, which is expressed as:

$${a}_{ISO} = f ( \frac{{e}_{c} * {C}_{u}}{P} , κ ) ;$$

a specific calculation formula for also is obtained by consulting tables for different bearings; a contamination coefficient e_{c} for automotive reducer bearings is selected between 0.8 to 0.9; a load fatigue limit Cᵤ is found from a supplier's website according to a bearing model; and a lubrication condition of the bearing is represented by a viscosity ratio κ, which is a ratio of an actual kinematic viscosity to a reference kinematic viscosity:

$$κ = \frac{v}{{v}_{1}} ;$$

v is the actual kinematic viscosity; the reference kinematic viscosity v₁ depends on a bearing rotational speed n and a pitch diameter D_{pw};
when n<1000 r/min, v₁=45000*n^{-0.83}*D_{pw}^{-0.5};
when n>1000 r/min, v₁=4500*n^{-0.5}*D_{pw}^{-0.5};
P is the equivalent dynamic load, which is calculated through the bearing force analysis:

$$P = X * {A}_{a} + Y * {A}_{r} ;$$

where Aᵣ is a radial force on a bearing, and Aₐ is an axial force on the bearing; by setting several groups of bearing input torque T, segment results of bearing forces are calculated using finite element simulation with Romax software, and simulation results are interpolated to obtain a fitting curve of the bearing forces relating to an input torque T; a test torque is substituted into the fitting curve to find a corresponding force result, thereby obtaining a radial force A, and an axial force Aₐ corresponding to each torque level; X and Y represent allocation coefficients which are combined with a torque-force matrix to obtain an equivalent dynamic load under each torque level.

In some embodiments, in step 3, a bearing life calculation formula under each rotational speed-torque condition is expressed as:

$${L}_{n m} = {a}_{1} {{a}_{ISO} ( \frac{{c}_{r}}{P} )}^{3} = {a}_{1} {{a}_{ISO} ( \frac{{c}_{r}}{{XA}_{a} + {YA}_{r}} )}^{3} = f ( T , n ) ;$$

f(T, n) represents that the bearing life under each rotational speed-torque condition is simplified as a function of the torque and rotational speed;
a bearing damage matrix before extrapolation at a 128×128 level is obtained based on calculated bearing life at each level and corresponding load frequencies at each level:

$${D}_{test} = \frac{{N}_{test}}{{L}_{nmtest}} ;$$

and
based on a bearing damage matrix of the original data, a load spectrum extrapolation is proceeded.

In some embodiments, during the extrapolation of the preprocessed data in step 4:
overall load spectrum extrapolation is performed by Gaussian kernel function combined with K strengthening coefficient;
load extremum extrapolation is performed by a load function method in a direct extrapolation of the K strengthening coefficient; a damage-frequency distribution of the original data is fitted and compared with various distributions, a distribution function with a smallest goodness-of-fit at a 90th percentile confidence level for each operating condition is selected; a distribution goodness-of-fit test method is used to verify an accuracy of an initially selected fitting distribution model, and Anderson-Darling test statistic is arranged to determine a goodness of fit:

$${AD}^{2} = {\int }_{- ∞}^{∞} {[ {F}_{n} ( x ) - F ( x ) ]}^{2} \frac{dF ( x )}{F ( x ) ( 1 - F ( x ) )} ;$$

a test method compares AD² with a critical value of a corresponding distribution cluster, a null hypothesis H₀ is accepted or rejected at a significance level α; Fₙ(x) represents a probability density distribution function of test data, and F(x) represents a probability density distribution function of a to-be-compared distribution, a smaller AD indicates better distribution fitting;
a probability density function corresponding to a data distribution is determined to obtain an inferred extreme value;
based on the inferred extreme value combined with a required extrapolation target reliability, a total damage D_{target} for a target life is determined; and the K strengthening coefficient is calculated by a formula:

$$K = \frac{{D}_{target}}{{D}_{test}} ;$$

and
wherein a total damage Dₜₑₛₜ of current test data is obtained from the bearing damage model in step 3.

In some embodiments, in step 4, an extrapolation process combining the Gaussian kernel function with the K strengthening coefficient is performed through steps of:
assuming that a random variable x follows an unknown probability density function \hat{f}(x), estimating a distribution of the unknown probability density function \hat{f}(x) using samples x₁, . . . , xₙ drawn from \hat{f}(x); defining a smooth function K(*) as a kernel function and h as a smoothing parameter which is bandwidth, wherein a bandwidth h=b−a is an assumed interval length, and assuming that the number of samples falling into an interval [a, b] is k, a relationship between the kernel function K(*) and the number of samples is expressed as:

$$k = {\sum }_{i = 1}^{n} K ( \frac{x - {x}_{i}}{h} ) ;$$

a probability density function \hat{f}(x) obtained based on a kernel density estimation principle is expressed as:

$$\hat{f} ( x ) = \frac{1}{nh} {\sum }_{i = 1}^{n} K ( \frac{x - {x}_{i}}{h} ) ;$$

a selected Gaussian kernel function is expressed as:

$$K = \frac{1}{\sqrt{2 π}} {e}^{- \frac{{x}^{2}}{2}} , x ∈ R ;$$

a corresponding two-dimensional kernel density estimation is expressed as:

$$\hat{f} ( x , y ) = \frac{1}{2 π n {h}^{2}} {\sum }_{i = 1}^{n} exp [ \frac{- {( x - {x}_{i} )}^{2}}{2 {h}^{2}} + \frac{- {( y - {y}_{i} )}^{2}}{2 {h}^{2}} ] ;$$

an adaptive bandwidth selection method is adopted through steps of:
introducing an adaptive factor λᵢ which is expressed as:

$${λ}_{i} = {( \frac{\hat{f} ( {u}_{i} )}{g} )}^{- 1 / 3} ;$$

wherein λᵢ is the adaptive factor, and \hat{f}(uᵢ) is a probability density value at a point uᵢ;
a calculation formula of g is expressed as:

$$lg ( g ) = \frac{1}{N} {\sum }_{i = 1}^{n} lg ( \hat{f} ( {u}_{i} ) ) ;$$

a final adaptive kernel density estimation is expressed as:

$$\hat{f} ( u ) = \frac{1}{{N ( {λ}_{i} * h )}^{2}} {\sum }_{i = 1}^{N} K ( \frac{x - {x}_{i}}{{λ}_{i} h} , \frac{y - {y}_{i}}{{λ}_{i} h} ) ;$$

N is the number of sample data;
by combining the K strengthening coefficient extrapolation and the Gaussian kernel function extrapolation, a set of extended frequency data under each rotational speed-torque level with expansions in both frequency and rotational speed-torque can be obtained:

$${N}_{extrapolation} = K * \hat{f} ( u ) .$$

In some embodiments, during a compilation of the multi-level program load spectrum in step 5, a frequency matrix corresponding to the combined rotational speed-torque counting method derives from two variables of a rotational speed and a torque;
based on an equivalent damage principle, the extrapolated user full-life cycle load spectrum under road conditions is reconstructed into a program loading spectrum as a loading input basis for durability or reliability testing; an extrapolated 128×128 level bearing damage obtained in step 4 is converted into an 8×8 level spectrum using an equivalent damage conversion principle; proportion coefficients for the 8×8 level spectrum are graded as 1, 0.95, 0.85, 0.725, 0.575, 0.425, 0.275, and 0.125; the equivalent damage conversion principle is expressed as:

$$D = {\sum }_{i = 1 , j = 1}^{i = l , j = l} \frac{{N}_{i , j}}{{L}_{i , j}} = \frac{{N}_{eq}}{{L}_{eq}} ;$$

wherein a damage at each level in the 8×8 level spectrum equals a sum of damages in a corresponding 128×128 level spectrum, Neg is a frequency corresponding to a set of rotational speed-torque in the 8×8 level spectrum, Leg is a bearing fatigue life corresponding to the set of rotational speed-torque in the 8×8 level spectrum, N_{i,j} is a frequency corresponding to a set of rotational speed-torque in the 128×128 level spectrum, L_{i,j} is a bearing fatigue life corresponding to the set of rotational speed-torque in the 128×128 level spectrum, and l denotes the number of subintervals contained in the 128 intervals corresponding to a certain level in the 8×8 level spectrum.

The beneficial effects of the present disclosure are as follows.

(1) During the load frequency extrapolation process, compared to traditional extrapolation methods, the present disclosure employs the extrapolation method combining Gaussian kernel function and K strengthening coefficient. This extrapolation method fully considers the distribution characteristics of the original load, leveraging the advantages of Gaussian kernel function in expanding the distribution range and K strengthening coefficient in linear frequency extrapolation. Consequently, the extrapolated load spectrum not only meets the required number of cycles for testing but also achieves reasonable expansion of load boundaries. This preserves load cycles that should exist but were not collected in the extrapolated load spectrum, enhancing the accuracy of load extrapolation.

(2) During the bearing force analysis process, the present disclosure performs piecewise interpolation calculation based on traditional bearing force analysis methods and combining with finite element software simulation results. The present disclosure also considers the influence of factors such as radial deformation of the bearing and the deflection deformation of the transmission shaft on the bearing force during analysis. The data obtained from finite element simulation enhances the alignment between the force analysis results and actual bearing forces. Meanwhile, fitting and interpolating the simulation results extends the finite simulation results to the entire torque cycle while saving testing time.

(3) According to the bearing life calculation formula, the rotational speed parameter influences the reference kinematic viscosity, thereby affecting the viscosity ratio, which in turn alters the magnitude of the coefficient a_{ISO} during life calculation. However, most current loading processes for bench bearing test spectra do not consider the effect of rotational speed. Therefore, the combined rotational speed-torque counting method employed in this disclosure can significantly enhance the accuracy and authenticity of bearing life analysis results.

### BRIEF DESCRIPTION OF THE DRAWINGS

The accompanying drawings, which are incorporated into and constitute a part of this specification, are intended to illustrate the present disclosure and explain the principles of the present disclosure in conjunction with the following specific embodiments but are not intended to limit the disclosure.

FIG. 1 is a flowchart of a multi-level bench spectrum construction method based on a combined rotational speed-torque counting method according to an embodiment of the present disclosure;

FIG. 2 is a schematic diagram showing a multi-level bench spectrum construction which records and stores each Ni with corresponding torque and rotational speed as a frequency matrix; and

FIG. 3 shows a final 8×8 level test spectrum regarding the bearing rotational speed and bearing torque, which is a required bearing bench test spectrum (in the figure, the torque is divided into 16 levels for ease of image analysis).

### DETAILED DESCRIPTION OF EMBODIMENTS

The present disclosure will be described in detail below in conjunction with the accompanying drawings and specific embodiments.

### Part One

Complete steps of the method in the present disclosure will be described.

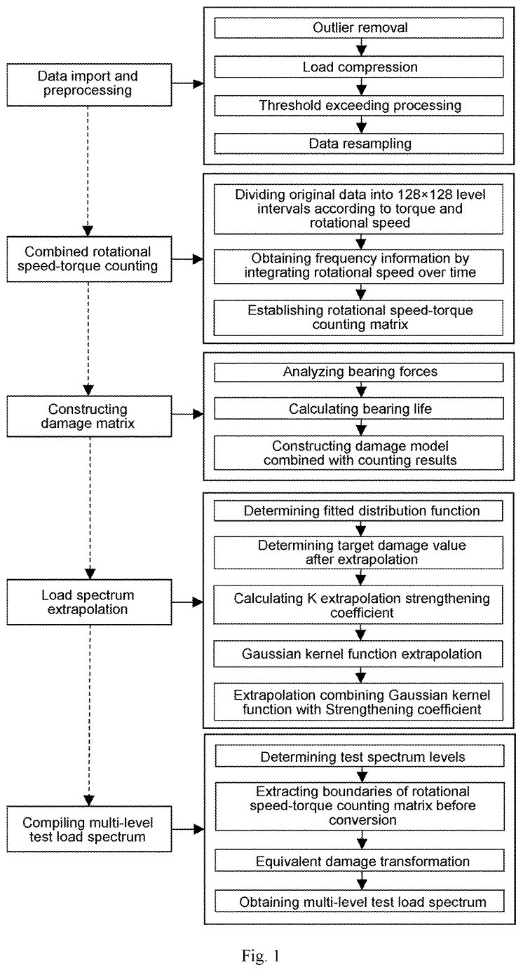

With reference to FIG. 1, the multi-level bench spectrum construction method based on the combined rotational speed-torque counting method proposed by the present disclosure primarily includes the following steps.

### Step 1: Acquiring and Preprocessing Sample Load Spectrum Road Operation Data

Original data directly collected from sensors may exhibit sudden changes, noise, and glitches due to the influence of driving environments, interference signals, and structures. Preprocessing operations such as outlier removal are required for the original data.

Outlier removal follows the PauTa Criterion. Considering that torque loads contain a large number of small torque fluctuation signals, which generate numerous low-amplitude, high-frequency load cycles, such loads generally do not cause fatigue damage but significantly increase computational load, resulting in data redundancy i.e., high-frequency noise. Therefore, such loads need to be eliminated. By performing Fourier transform on the original data, the original frequency spectrum can be obtained. Through Butterworth filtering, torque data under various amplitudes can be screened according to frequency size and categorized into primary waves, secondary waves, and tertiary waves. By removing the tertiary waves with low amplitude and high frequency (amplitude data segments less than ±1 Nm), preliminary filtering of invalid waves can be accomplished.

Threshold exceeding processing refers to filling missing data using piecewise interpolation after removing abnormal data outside the required rotational speed-torque range.

Data resampling is conducted because the sampling frequencies of various signals are inconsistent, and the data points of each signal cannot correspond one-to-one in terms of time. Therefore, to enable joint distribution statistics of all signals, resampling is performed on all data to unify the sampling frequency. Resampling is performed at a specified frequency of 10 Hz through interpolation, extracting non-redundant time points and interpolating at a fixed, uniform sampling frequency.

### Step 2: Combined Rotational Speed-Torque Counting on the Original Data

The data under each rotational speed-torque condition is counted by dividing the preprocessed time-domain data into intervals (typically dividing each of torque and rotational speed into 128 levels), and the rotational speed within each small rotational speed-torque interval is integrated over time:

$${N}_{i} = {\sum }_{j = 1}^{m} {\int }_{{Δ}_{{t}_{i}}} n ( i ) dt .$$

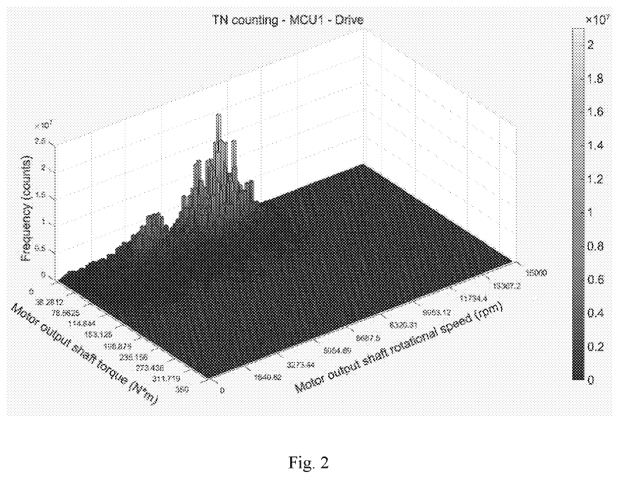

The frequency Ni under each rotational speed-torque interval is recorded. Each Nᵢ along with its corresponding rotational speed-torque values are saved as a frequency matrix. The result is shown in FIG. 2.

### Step 3: Damage Model Construction

During the calculation of bearing damage at each rotational speed-torque level, the bearing damage at each level equals the loading frequency N at each level divided by the corresponding bearing life Lₙₘ, which is expressed as:

$$D = \frac{N}{{L}_{n m}} .$$

Based on step 2, the loading frequencies corresponding to each rotational speed-torque level before extrapolation are obtained.

The bearing life before extrapolation is provided in “GB/T 6391-2010 Rolling Bearings-Dynamic Load Ratings and Rating Life”, as expressed by:

$${L}_{n m} = {a}_{1} ⋆ {{a}_{I S O} ( \frac{{C}_{r}}{P} )}^{m} .$$

In above formula, Cr represents the basic dynamic load rating, which can be directly obtainable according to the bearing model; m denotes the bearing life exponent, where m=3 for ball bearings and m=10/3 for roller bearings; a₁ serves as the reliability correction coefficient; P refers to the equivalent dynamic load; and a_{ISO} is a correction coefficient that is a function of rotational speed, while also being related to factors such as the bearing's contamination coefficient, load fatigue limit, and lubrication conditions:

$${a}_{I S O} = f ( \frac{{e}_{c} ⋆ {C}_{u}}{P} , κ ) .$$

The specific calculation formula for a_{ISO} can be obtained by consulting tables for different bearing selections. According to reference materials: the contamination factor e_{c} for automotive reducer bearings is typically selected as 0.8 to 0.9, the load fatigue limit Cᵤ can be found on the supplier's official website based on the bearing model; the lubrication condition of the bearing is represented by the viscosity ratio K, i.e., the ratio of actual kinematic viscosity to reference kinematic viscosity:

$$κ = \frac{v}{{v}_{1}} .$$

Actual kinematic viscosity v relates to lubricant properties, rotational speed, and operating temperature during bearing operation. The characteristics of lubricants refer to the viscosity grades, which are defined by specific standards in ISO. The actual viscosity of lubricants can be determined by tables or calculations based on the viscosity grade and operating temperature.

The reference kinematic viscosity v₁ depends on the bearing rotational speed n and the pitch diameter D_{pw}, and is calculated by the following formula.

When n<1000 r/min, v₁=45000*n^{-0.83}*D_{pw}^{-0.5}.

When n>1000 r/min, v₁=4500*n^{-0.5}*D_{pw}^{-0.5}.

P is the equivalent dynamic load, which is calculated through the bearing force analysis:

$$P = X ⋆ {A}_{a} + Y ⋆ {A}_{r} .$$

In above formula, Aᵣ is the radial force on the bearing, and Aₐ is the axial force on the bearing. By setting several groups of bearing input torque T, segment results of bearing forces are calculated using finite element simulation using Romax software, and simulation results are interpolated to obtain the fitting curve of the bearing forces versus the input torque T. The test torque is substituted into the fitting curve to find the corresponding force result, thereby obtaining the radial force Aᵣ and the axial force Aₐ corresponding to each torque level. X and Y represent allocation coefficients, which can be determined by referring to the standard GB/T 6391-2010. By combining the torque-force matrix, the equivalent dynamic load under each torque level can be obtained.

The final bearing life calculation formula for each rotational speed-torque condition can be simplified as:

$${L}_{n m} = {a}_{1} {{a}_{I S O} ( \frac{{C}_{r}}{P} )}^{3} = {a}_{1} {{a}_{I S O} ( \frac{{C}_{r}}{X {A}_{a} + Y {A}_{r}} )}^{3} = f ( T , n ) .$$

The bearing damage matrix before extrapolation at the 128×128 level is obtained based on final calculated bearing life at each level and corresponding load frequencies at each level:

$${D}_{t e s t} = \frac{{N}_{t e s t}}{{L}_{n m t e s t}} .$$

Based on the bearing damage matrix of the original data, the next step of the load spectrum extrapolation will be proceeded.

### Step 4: Extrapolation on the Original Data

The load spectrum extrapolation is performed using the extrapolation method that combines Gaussian kernel function with K strengthening coefficient.

### Step 4.1: K Strengthening Coefficient Extrapolation

The extrapolation based on the K strengthening coefficient requires the damage values before and after extrapolation, as follows:

$$K = \frac{{D}_{target}}{{D}_{t e s t}} .$$

That is, the extrapolation factor=total damage for target lifetime/total damage from current test data.

The acquisition of the target damage D_{target} requires the load extremum extrapolation.

The load extremum extrapolation selects the load function method. The damage-frequency distribution of the original data is fitted and compared with various distributions, the distribution function with the smallest goodness-of-fit at the 90th percentile confidence level for each operating condition is selected. The distribution goodness-of-fit test method is used to verify the accuracy of an initially selected fitting distribution model, and the Anderson-Darling test statistic is used to determine the goodness of fit:

$$A {D}^{2} = {\int }_{- ∞}^{∞} {[ {F}_{n} ( x ) - F ( x ) ]}^{2} \frac{dF ( x )}{F ( x ) ( 1 - F ( x ) )} .$$

This test method compares AD² with the critical value of the corresponding distribution cluster. At a significance level α, a null hypothesis H₀ is accepted or rejected. Fₙ(x) represents a probability density distribution function of test data, and F(x) represents a probability density distribution function of a to-be-compared distribution. A smaller AD value indicates better distribution fitting.

Here, the user damage per unit mileage is used as the intensity indicator, which should represent the original load damage intensity per unit mileage of the 90th percentile users. By fitting the Weibull distribution probability curve using the original damage per unit mileage as input, the value corresponding to P=0.9 on the probability curve is taken as the target damage per unit mileage for the 90th percentile users, denoted as d₉₀. Combined with the target service life mileage L_{target}, the overall target damage for the vehicle's full life cycle is calculated: D_{target}=d₉₀×L_{target}.

Based on step 3, Dₜₑₛₜ can be calculated.

Therefore, the K strengthening coefficient can be obtained by substituting the overall target damage D_{target} for the full vehicle life cycle and the test damage Dₜₑₛₜ

$$K = \frac{{D}_{target}}{{D}_{t e s t}} .$$

### Step 4.2: Extrapolation Process of Gaussian Kernel Function

Assume that the distribution of the corresponding values x of rotational speed and torque at each level a random variable follows an unknown probability density function \hat{f}(x), distribution of the function \hat{f}(x) is estimated using samples x₁, . . . , xₙ drawn from \hat{f}(x). The smooth function K(*) is defined as the kernel function, and h is a smoothing parameter (bandwidth). The bandwidth h=b−a is an assumed interval length, and assuming that the number of samples falling into the interval [a, b] is k, the relationship between the kernel function K(*) and the number of samples is expressed as:

$$k = {\sum }_{i = 1}^{n} K ( \frac{x - {x}_{i}}{h} ) .$$

The probability density function \hat{f}(x) obtained based on a kernel density estimation principle is expressed as:

$$\hat{f} ( x ) = \frac{1}{nh} {\sum }_{i = 1}^{n} K ( \frac{x - {x}_{i}}{h} ) .$$

The selected Gaussian kernel function is expressed as:

$$K = \frac{1}{\sqrt{2 π}} {e}^{- \frac{{x}^{2}}{2}} , x ∈ R .$$

The corresponding two-dimensional kernel density estimation is expressed as:

$$\hat{f} ( x , y ) = \frac{1}{2 π {nh}^{2}} {\sum }_{i = 1}^{n} exp [ \frac{- {( x - {x}_{i} )}^{2}}{2 {h}^{2}} + \frac{- {( y - {y}_{i} )}^{2}}{2 {h}^{2}} ]$$

Regarding the bandwidth that is another factor influencing the probability density function under the principle of kernel density estimation, there exists an optimal bandwidth selection. A larger bandwidth coefficient yields a smoother estimated density function curve, but may result in underfitting, meaning certain characteristics of the original sample data may be obscured. A smaller bandwidth coefficient may cause the estimated density function curve to overfit, resulting in increased variance.

The adaptive bandwidth is selected as follows.

An adaptive factor λᵢ is introduced and expressed as:

$${λ}_{i} = {( \frac{\hat{f} ( {u}_{i} )}{g} )}^{- 1 / 3} .$$

In above formula, λᵢ is the adaptive factor, and \hat{f}(uᵢ) is a probability density value at a point uᵢ.

The calculation formula of g is expressed as:

$$lg ( g ) = \frac{1}{N} {\sum }_{i = 1}^{n} lg ( \hat{f} ( {u}_{i} ) ) .$$

The final adaptive kernel density estimation is expressed as:

$$\hat{f} ( u ) = \frac{1}{{N ( {λ}_{i} * h )}^{2}} {\sum }_{i = 1}^{n} K ( \frac{x - {x}_{i}}{{λ}_{i} h} , \frac{y - {y}_{i}}{{λ}_{i} h} ) .$$

N is the number of sample data. Substituting the torque and rotational speed used for estimation into the above x and y yields the adaptive kernel density estimate result.

The kernel density estimation with the adaptive factor allows the bandwidth to vary with data density—narrowing in dense regions and widening in sparse areas. This yields probability densities closer to the actual distribution compared to traditional fixed-bandwidth methods.

By combining the K strengthening coefficient extrapolation and the Gaussian kernel function extrapolation, a set of extended frequency data under each rotational speed-torque level with expansions in both the frequency and rotational speed-torque can be obtained:

$${N}_{extrapolation} = K * \hat{f} ( u ) .$$

### Step 5: Compiling a Multi-Level Program Load Spectrum

Torque and rotational speed are each divided into 8 levels according to specific proportions. The extrapolated rotational speed-torque frequency matrix from step 4 is processed using the Palmgren-Miner rule combined with the damage equivalence principle. According to an upward equivalence method, the frequencies within the 128×128 level small intervals are transformed into frequencies over the 8×8 level large intervals. The proportion coefficients for the 8 levels are graded as follows: 1, 0.95, 0.85, 0.725, 0.575, 0.425, 0.275, and 0.125.

The equivalent damage conversion principle is expressed as:

$$D = {\sum }_{i = 1 , j = 1}^{i = l , j = l} \frac{{N}_{i , j}}{{L}_{i , j}} = \frac{{N}_{eq}}{{L}_{eq}} .$$

The damage at each level in the 8×8 level spectrum equals the sum of damages in the corresponding 128×128 level spectrum. N_{eq} represents the frequency corresponding to a set of rotational speed-torque in the 8×8 level spectrum, and L_{eq} represents the bearing fatigue life corresponding to a set of rotational speed-torque in the 8×8 level spectrum. N_{i,j} represents the frequency corresponding to a set of rotational speed-torque in the 128×128 level spectrum, and L_{i,j} represents the bearing fatigue life corresponding to a set of rotational speed-torque in the 128×128 level spectrum. l denotes the number of subintervals contained in the 128 intervals corresponding to a certain level in the 8×8 level spectrum.

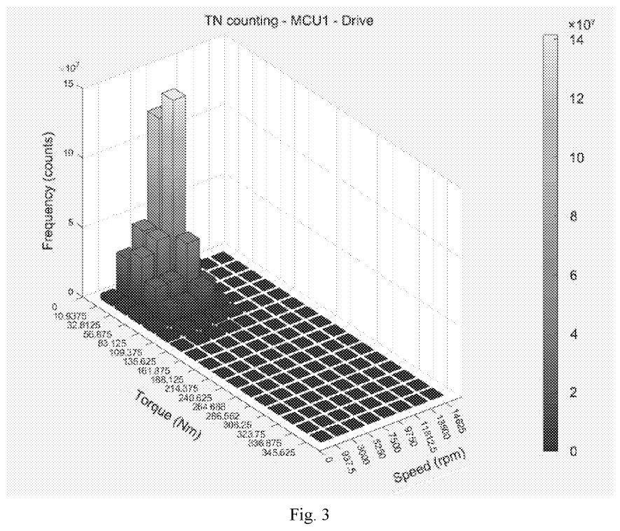

The final 8×8 level test spectrum in terms of the bearing rotational speed and torque constitutes the required bearing bench test spectrum. As shown in FIG. 3, the torque is divided into 16 levels for easier graphical analysis.

### Part Two

Theoretical Analysis of the disclosure's Feasibility.

(1) During the load frequency extrapolation process, compared to traditional extrapolation methods, the present disclosure employs the extrapolation method combining Gaussian kernel function and K strengthening coefficient. This extrapolation method fully considers the distribution characteristics of the original load, leveraging the advantages of Gaussian kernel function in expanding the distribution range and K strengthening coefficient in linear frequency extrapolation. Consequently, the extrapolated load spectrum not only meets the required number of cycles for testing but also achieves reasonable expansion of load boundaries. This preserves load cycles that should exist but are not collected in the extrapolated load spectrum, enhancing the accuracy of load extrapolation.

(2) During the bearing force analysis process, the present disclosure performs piecewise interpolation calculation based on traditional bearing force analysis methods and combining with finite element software simulation results. The present disclosure also considers the influence of factors such as radial deformation of the bearing and the deflection deformation of the transmission shaft on the bearing force during analysis. The data obtained from finite element simulation enhances the alignment between the force analysis results and actual bearing forces. Meanwhile, fitting and interpolating the simulation results extends the finite simulation results to the entire torque cycle while saving testing time.

(3) According to the bearing life calculation formula, the rotational speed parameter influences the reference kinematic viscosity, thereby affecting the viscosity ratio, which in turn alters the magnitude of the coefficient a_{ISO} during life calculation. However, most existing loading processes for bench bearing test spectra do not consider the effect of rotational speed. Therefore, the combined rotational speed-torque counting method employed in this disclosure can significantly enhance the accuracy and authenticity of bearing life analysis results.

In this embodiment of the present disclosure, step 1 involves preprocessing the speed and torque characteristic parameter data collected by sensors. Based on this, steps 2-5 jointly count the frequencies of torque and rotational speed under various operating conditions according to different vehicle driving states. Then, the distribution of rotational speed and torque under the expected life is then extrapolated through Gaussian kernel function combined with the K strengthening coefficient. Both torque and rotational speed—parameters affecting the reliability and durability of reducer bearings—are jointly represented in space. Subsequently, the Palmgren-Miner rule, combined with the equivalent damage conversion method and the upward equivalence method is applied to transform the distribution into the bench test spectrum. This not only achieves accurate and reasonable extrapolation of the load spectrum obtained from testing but also significantly enhances the accuracy of the life analysis. Concurrently, the three-dimensional load spectrum image facilitates more intuitive and efficient analysis of reliability and durability.

Described above are merely embodiments of the disclosure, which are not intended to limit the disclosure. It should be understood that any modifications and replacements made by those skilled in the art without departing from the spirit of the disclosure should fall within the scope of the disclosure defined by the appended claims.

What is claimed is:

1. A method for constructing a bearing bench test spectrum based on a combined rotational speed-torque counting method, comprising:
step 1: acquiring and preprocessing sample load spectrum road operation data, and extracting preprocessed time-domain data;
step 2: performing combined rotational speed-torque counting on the preprocessed time-domain data: recording loading frequencies under each rotational speed-torque interval through interval counting;
step 3: constructing a bearing damage model: conducting bearing force analysis, calculating an equivalent dynamic load based on analysis results, and combining a correction coefficient a_{ISO} considering rotational speed parameters to obtain a bearing life calculation formula with a bearing torque and a bearing rotational speed as independent variables, and combining joint counting results to establish the bearing damage model;
step 4: performing extrapolation on preprocessed data: based on results of the bearing damage model and extrapolated bearing target damage, obtaining a K strengthening coefficient, deriving a probability density function via kernel density estimation, and extrapolating counting results obtained in step 2 by combining Gaussian kernel function with the K strengthening coefficient to obtain an extrapolated frequency matrix for the torque and rotational speed; and
step 5: compiling a multi-level program load spectrum: dividing each of the torque and rotational speed into eight levels according to specific ratios; applying a Palmgren-Miner rule in conjunction with a material S-N curve to the extrapolated rotational speed-torque frequency matrix from step 4, converting, by an upward equivalence method, frequencies within a 128×128 level interval to frequencies on a 8×8 level interval based on an equivalent damage conversion principle to obtain a 8×8 level bearing bench test spectrum for the torque and rotational speed.

2. The method of claim 1, wherein the preprocessing in step 1 comprises outlier removal, threshold exceeding processing, and data resampling;
the outlier removal comprises: performing Fourier transform on original data to obtain an original frequency spectrum, filtering through Butterworth filter to screen torque data under each amplitude based on a frequency size, following classifying into a primary wave, a secondary wave, and a tertiary wave; and removing tertiary waves with amplitude data segments less than ±1 Nm to preliminarily filter out invalid waves;
the threshold exceeding processing is to fill missing data using piecewise interpolation after performing the outlier removal; and
the data resampling comprises: resampling by interpolation at a specified frequency over time, extracting non-repeating time points, and interpolating at a fixed, uniform sampling frequency to obtain the preprocessed time-domain data.

3. The method of claim 1, wherein during the combined rotational speed-torque counting in step 2, the preprocessed time-domain data is first divided into 128×128 level intervals according to a target rotational speed-torque interval for counting, a rotational speed n(t) within a i-th rotational speed-torque interval is integrated over time t, time for each interval is Δtᵢ, and the number of torque segments within each interval is m, an integration result is recorded as a frequency Nᵢ for this interval, and frequencies for all intervals are stored as a matrix:
$${N}_{i} = {\sum }_{j = 1}^{m} {\int }_{{Δ}_{{t}_{i}}} n ( t ) dt .$$

4. The method of claim 1, wherein in the process of constructing the bearing damage model in step 3, a bearing damage D is calculated by a formula:
$$D = \frac{N}{{L}_{nm}} ;$$
wherein N represents a loading frequency at each level, and Lₙₘ represents a corresponding bearing life at each level.

5. The method of claim 1, wherein the loading frequencies corresponding to each rotational speed-torque level before extrapolation are obtained from step 2;
a bearing life before extrapolation:
$${L}_{nm} = {a}_{1} * {{a}_{ISO} ( \frac{{C}_{r}}{P} )}^{m} ;$$
wherein Cᵣ is a basic dynamic load rating; m is a bearing life exponent, for a ball bearing, m=3, and for a roller bearing,
$$m = \frac{10}{3} ;$$
a₁ is a reliability correction coefficient; P is an equivalent dynamic load; the correction coefficient a_{ISO} is a function of rotational speed, which is expressed as:
$${a}_{ISO} = f ( \frac{{e}_{c} * {C}_{u}}{P} , κ ) ;$$
a calculation formula for a_{ISO} is obtained by consulting tables for different bearings; a contamination coefficient e_{c} for automotive reducer bearings is selected between 0.8 to 0.9; a load fatigue limit Cᵤ is found from a supplier's website according to a bearing model; and a lubrication condition of the bearing is represented by a viscosity ratio κ, which is a ratio of an actual kinematic viscosity to a reference kinematic viscosity:
$$κ = \frac{v}{{v}_{1}} ;$$
v is the actual kinematic viscosity; the reference kinematic viscosity v₁ depends on a bearing rotational speed n and a pitch diameter D_{pw};
when n<1000 r/min,
$${v}_{1} = 45000 * {n}^{- 0.83} * {D}_{pw}^{- 0.5} ;$$
when n>1000 r/min,
$${v}_{1} = 4500 * {n}^{- 0.5} * {D}_{p w}^{- 0.5} ;$$
P is the equivalent dynamic load, which is calculated through the bearing force analysis:
$$P = X * {A}_{a} + Y * {A}_{r} ;$$
where Aᵣ is a radial force on a bearing, and Aₐ is an axial force on the bearing; by setting several groups of bearing input torque T, segment results of bearing forces are calculated using finite element simulation with Romax software, and simulation results are interpolated to obtain a fitting curve of the bearing forces relating to an input torque T; a test torque is substituted into the fitting curve to find a corresponding force result, thereby obtaining a radial force Aᵣ and an axial force Aₐ corresponding to each torque level; X and Y represent allocation coefficients which are combined with a torque-force matrix to obtain an equivalent dynamic load under each torque level.

6. The method of claim 5, wherein in step 3, a bearing life calculation formula under each rotational speed-torque condition is expressed as:
$${L}_{n m} = {a}_{1} {{a}_{ISO} ( \frac{{C}_{r}}{P} )}^{3} = {a}_{1} {{a}_{ISO} ( \frac{{C}_{r}}{{XA}_{a} + {YA}_{r}} )}^{3} = f ( T , n ) ;$$
f(T, n) represents that the bearing life under each rotational speed-torque condition is simplified as a function of the torque and rotational speed;
a bearing damage matrix before extrapolation at a 128×128 level is obtained based on calculated bearing life at each level and corresponding load frequencies at each level:
$${D}_{test} = \frac{{N}_{test}}{{L}_{nmtest}} ;$$
and
based on a bearing damage matrix of the original data, a load spectrum extrapolation is proceeded.

7. The method of claim 1, wherein during the extrapolation of the preprocessed data in step 4:
overall load spectrum extrapolation is performed by Gaussian kernel function combined with K strengthening coefficient;
load extremum extrapolation is performed by a load function method in a direct extrapolation of the K strengthening coefficient; a damage-frequency distribution of the original data is fitted and compared with various distributions, a distribution function with a smallest goodness-of-fit at a 90th percentile confidence level for each operating condition is selected; a distribution goodness-of-fit test method is used to verify an accuracy of an initially selected fitting distribution model, and Anderson-Darling test statistic is arranged to determine a goodness of fit:
$$A {D}^{2} = {\int }_{- ∞}^{∞} {[ {F}_{n} ( x ) - F ( x ) ]}^{2} \frac{dF ( x )}{F ( x ) ( 1 - F ( x ) )} ;$$
a test method compares AD² with a critical value of a corresponding distribution cluster, a null hypothesis H₀ is accepted or rejected at a significance level α; Fₙ(x) represents a probability density distribution function of test data, and F(x) represents a probability density distribution function of a to-be-compared distribution, a smaller AD indicates better distribution fitting;
a probability density function corresponding to a data distribution is determined to obtain an inferred extreme value;
based on the inferred extreme value combined with a required extrapolation target reliability, a total damage D_{target} for a target life is determined; and the K strengthening coefficient is calculated by a formula:
$$K = \frac{{D}_{target}}{{D}_{test}} ;$$
and
wherein a total damage Dₜₑₛₜ of current test data is obtained from the bearing damage model in step 3.

8. The method of claim 1, wherein in step 4, an extrapolation process combining the Gaussian kernel function with the K strengthening coefficient is performed through steps of:
assuming that a random variable x follows an unknown probability density function \hat{f}(x), estimating a distribution of the unknown probability density function \hat{f}(x) using samples x₁, . . . , xₙ drawn from \hat{f}(x); defining a smooth function K(*) as a kernel function and h as a smoothing parameter which is bandwidth, wherein a bandwidth h=b−a s is an assumed interval length, and assuming that the number of samples falling into an interval [a, b] is k, a relationship between the kernel function K(*) and the number of samples is expressed as:
$$k = {\sum }_{i = 1}^{n} K ( \frac{x - {x}_{i}}{h} ) ;$$
a probability density function \hat{f}(x) obtained based on a kernel density estimation principle is expressed as:
$$\hat{f} ( x ) = \frac{1}{n h} {\sum }_{i = 1}^{n} K ( \frac{x - {x}_{i}}{h} ) ;$$
a selected Gaussian kernel function is expressed as:
$$K = \frac{1}{\sqrt{2 π}} {e}^{- \frac{{x}^{2}}{2}} , x ∈ R ;$$
a corresponding two-dimensional kernel density estimation is expressed as:
$$\hat{f} ( x , y ) = \frac{1}{2 π n {h}^{2}} {\sum }_{i = 1}^{n} exp [ \frac{- {( x - {x}_{i} )}^{2}}{2 {h}^{2}} + \frac{- {( y - {y}_{i} )}^{2}}{2 {h}^{2}} ] ;$$
an adaptive bandwidth selection method is adopted through steps of:
introducing an adaptive factor λᵢ which is expressed as:
$${λ}_{i} = {( \frac{\hat{f} ( {u}_{i} )}{g} )}^{- 1 / 3} ;$$
wherein λᵢ is the adaptive factor, and \hat{f}(uᵢ) is a probability density value at a point uᵢ;
a calculation formula of g is expressed as:
$$lg ( g ) = \frac{1}{N} {\sum }_{i = 1}^{n} lg ( \hat{f} ( {u}_{i} ) ) ;$$
a final adaptive kernel density estimation is expressed as:
$$\hat{f} ( u ) = \frac{1}{{N ( {λ}_{i} * h )}^{2}} {\sum }_{i = 1}^{N} K ( \frac{x - {x}_{i}}{{λ}_{i} h} , \frac{y - {y}_{i}}{{λ}_{i} h} ) ;$$
N is the number of sample data;
by combining K strengthening coefficient extrapolation and Gaussian kernel function extrapolation, a set of extended frequency data under each rotational speed-torque level with expansions in both the frequency and rotational speed-torque is obtained:
$${N}_{extrapolation} = K * \hat{f} ( u ) .$$

9. The method of claim 1, wherein during a compilation of the multi-level program load spectrum in step 5, a frequency matrix corresponding to the combined rotational speed-torque counting method derives from two variables of a rotational speed and a torque;
based on an equivalent damage principle, the extrapolated user full-life cycle load spectrum under road conditions is reconstructed into a program loading spectrum as a loading input basis for durability or reliability testing; an extrapolated 128×128 level bearing damage obtained in step 4 is converted into an 8×8 level spectrum using an equivalent damage conversion principle; proportion coefficients for the 8×8 level spectrum are graded as 1, 0.95, 0.85, 0.725, 0.575, 0.425, 0.275, and 0.125; the equivalent damage conversion principle is expressed as:
$$D = {\sum }_{i = 1 , j = 1}^{i = l , j = l} \frac{{N}_{i , j}}{{L}_{i , j}} = \frac{{N}_{eq}}{{L}_{eq}} ;$$
wherein a damage at each level in the 8×8 level spectrum equals a sum of damages in a corresponding 128×128 level spectrum, N_{eq} is a frequency corresponding to a set of rotational speed-torque in the 8×8 level spectrum, L_{eq} is a bearing fatigue life corresponding to the set of rotational speed-torque in the 8×8 level spectrum, N_{i,j} is a frequency corresponding to a set of rotational speed-torque in the 128×128 level spectrum, L_{i,j} is a bearing fatigue life corresponding to the set of rotational speed-torque in the 128×128 level spectrum, and l denotes the number of subintervals contained in 128 intervals corresponding to a certain level in the 8×8 level spectrum.
